(19) United States
(12) Patent Application Publication
(10) Pub. No.: US 2024/0339214 A1
(43) Pub. Date: Oct. 10, 2024

(54) METHOD AND SYSTEM FOR OPTIMIZING MEDICAL IMAGINING EXAMINATION

(71) Applicant: L&T TECHNOLOGY SERVICES LIMITED, Chennai (IN)

(72) Inventor: HAJA NAZURUDEEN MOHAMED SHEIK KATHI, Thanjavur District (IN)

(21) Appl. No.: 18/209,533

(22) Filed: Jun. 14, 2023

(30) Foreign Application Priority Data
Apr. 4, 2023 (IN) 202341025496

(51) Int. Cl. G16H 50/20; G16H 40/63

(52) U.S. Cl. CPC G16H 50/20; G16H 40/63; G16H 10/60; G16H 70/20

(57) ABSTRACT

The present invention describes an artificial intelligence (Al) based protocol selection method. The method recites identifying user physical information, selecting one or more medical examination protocols based on at least a part of the identified user physical information, identifying a plurality of protocol parameters for the medical examination, based on the selected one or more medical examination protocols, receiving input data, processing the received input data to change the at least one protocol parameter, and providing the plurality of protocol parameters based on the processed received input data to a medical imaging apparatus for performing the medical imaging examination of the user.

### TECHNICAL FIELD

The present invention generally relates to an artificial intelligence (Al) based protocol selection techniques for optimizing medical imaging examination performed by an imaging apparatus.

### BACKGROUND

The following description includes information that may be useful in understanding the present invention. It is not an admission that any of the information provided herein is prior art or relevant to the presently claimed invention, or that any publication specifically or implicitly referenced is prior art.

Medical imaging apparatuses usually have several components, control of which are executed by software that causes algorithms to be executed. These medical imaging apparatuses generate image data of a user or other subject for examination. Based on the image data, clinical diagnoses are created, and diseases are diagnosed. Further, depending on the clinical objective, control parameters for controlling the medical imaging apparatus are selected. These control parameters, which are usually large in number, are collectively referred as protocol. For the examination of the user, it is necessary for the medical imaging apparatus to be controlled by a number of individually distinct protocols, depending upon the clinical objective.

For controlling a medical imaging apparatus, typically one protocol can be selected among a number of protocols and customized to the examination to be executed on the user. In order to select the number of protocols, a technician or physician first performs manual assessment of the user and selects the protocols manually based on the manual assessment of the user. After the selection of the protocols, the technician or physician fill the details regarding protocol parameters of the selected protocols and able to change the protocol parameters in order to perform medical examination of the user by the imaging apparatus.

Conventional procedures related to selection of protocols and protocol parameters for medical imaging are performed by the technician or physician. Thus, there are high chances of error in selection of protocol by technician or physician which will hamper the examination procedure performed by an imaging apparatus.

Further, the conventional procedures are time consuming procedure which will hamper the health of the user in case of medical emergency. For example, if a user comes to a hospital during an emergency condition, the first step the physician will perform is a preliminary check of the patient. After the preliminary check, the physician may suggest a medical imaging examination. In case of a suggestion for a medical imaging examination, the physician or technician will first identify the protocols and their parameters before starting the medical imaging examination. After identifying the protocols and their parameters manually, the medical imaging examination will take place. However, this manual procedure is a time-consuming process in order to perform the medical examination of the user, and this may harm the health of the user.

Thus, there is a need for a system that is able to select a protocol automatically and identifies the protocol parameters corresponding to the selected protocol in order to optimize medical imaging examination procedure that is to be performed on the user.

### SUMMARY

The present disclosure overcomes one or more shortcomings of the prior art and provides additional advantages discussed throughout the present disclosure. Additional features and advantages are realized through the techniques of the present disclosure. Other embodiments and aspects of the disclosure are described in detail herein and are considered a part of the claimed disclosure.

In an aspect, the present disclosure recites an artificial intelligence (Al) based protocol selection method for optimizing medical imaging examination of a user. The method includes identifying user physical information. The user physical information includes at least one of gesture, height, weight, injury, motion, and structure information of the user. The method further includes retrieving medical information of the user, the medical information of the user corresponds to at least one of a medical history and current medical assessment of the user. The method further includes selecting one or more medical examination protocols based on at least a part of the identified user physical information and identifying a plurality of protocol parameters for the medical examination, based on the retrieved user medical information and the selected one or more medical examination protocols. The method further includes receiving input data tagged with a priority level. The received input data is related to a change in at least one protocol parameter among the plurality of protocol parameters. The method further includes processing the received input data to change the at least one protocol parameter based on at least one of the priority level and a pre-defined range of the at least one protocol parameter. Furthermore, the method includes providing the plurality of protocol parameters based on the processed input data to a medical imaging apparatus for performing the medical imaging examination of the user.

In another aspect, the present disclosure recites a method, for identifying the plurality of protocol parameters for optimizing medical imaging examination of the user that includes performing region of interest projection on the identified user physical information. The identified user physical information corresponds to imaging data, received in form of an image or a video, of the user. The method further discloses identifying region of interest based on the region of interest projection, Further, the method discloses determining demographic information of the user based on the identified region of interest and identifying the plurality of protocol parameters for the medical examination based on the retrieved medical information and the determined demographic information.

In another aspect, the present disclosure recites a method for selecting the one or more medical examination protocols for optimizing medical imaging examination of the user that includes performing region of interest projection on the identified user physical information. The identified user physical information corresponds to imaging data, received in the form of an image or a video, of the user. The method further discloses extracting the at least one of gesture, injury, and motion information from the identified user physical information based on the region of interest projection. The method further discloses selecting the one or more medical examination protocols based on the extracted at least one of gesture, injury, and motion information.

In another aspect, the present disclosure recites a method for processing the received input data for optimizing medical imaging examination of the user that includes extracting, from the received input data, one or more protocol parameters and features corresponding to the one or more protocol parameters. The method further discloses identifying the at least one protocol parameter among the plurality of protocol parameters based on the extracted features. The method further discloses controlling the change in the at least one protocol parameter with respect to the extracted one or more protocol parameters, based on the at least one of priority level and suggested change falling within the pre-defined range of the at least one protocol parameter.

In another aspect, the present disclosure recites a method for processing the received input data for optimizing medical imaging examination of the user that includes when the priority level is indicated high, changing the at least one protocol parameter with respect to the extracted one or more protocol parameters whether the suggested change falls within the pre-defined range or outside the pre-defined range. The method further discloses when the priority level is indicated low, controlling the change in the at least one protocol parameter with respect to the extracted one or more protocol parameters based on the medical information whether the suggested change falls within the pre-defined range or outside the pre-defined range.

In an aspect, the present disclosure recites an artificial intelligence (AI) based protocol selection system for optimizing a medical imaging examination of a user. The system comprises an imaging and sensing unit configured to identify user physical information. The user physical information includes at least one of gesture, height, weight, injury, motion, and structure information of the user. The system further includes one or more processor operatively coupled to the imaging and sensing unit, the one or more processor is configured to: retrieve medical information of the user. The user medical information corresponds to at least one of a medical history and current medical assessment of the user. The one or more processor is further configured to select one or more medical examination protocols based on at least a part of the identified user physical information. The one or more processor is further configured to identify a plurality of protocol parameters for the medical examination, based on the retrieved user medical information and the selected one or more medical examination protocols. Furthermore, the system includes a receiving unit operatively coupled to the one or more processor and configured to receive input data tagged with priority level. The received input data is related to a change in at least one protocol parameter among the plurality of protocol parameters. The one or more processor is further configured to process the received input to change the at least one protocol parameter based on at least one of the priority level and a pre-defined range of the at least one protocol parameter. Furthermore, the system comprises a transmitting unit operatively coupled to the one or more processor and configured to provide the plurality of protocol parameters based on the processed received input data to a medical imaging apparatus for performing the medical imaging examination of the user.

In another aspect, the present disclosure recites one or more processor to identify the plurality of parameters for optimizing a medical imaging examination of a user. The one or more processor is configured to perform region of interest projection on the identified user physical information. The identified user physical information corresponds to imaging data, received in form of an image or a video, of the user, identify region of interest based on the region of interest projection. The one more processor is further configured to determine demographic information of the user based on the identified region of interest. The one or more processor is further configured to identify the plurality of protocol parameters for the medical examination based on the retrieved medical information and the determined demographic information.

In another aspect, the present disclosure recites one or more processor to select the one or more medical examination protocols for optimizing a medical imaging examination of a user. The one or more processor is configured to perform region of interest projection on the identified user physical information. The identified user physical information corresponds to imaging data, received in the form of an image or a video, of the user. The one or more processor is further configured to extract the at least one of gesture, injury, and motion information from the identified user physical information based on the region of interest projection. The one or more processor is further configured to select the one or more medical examination protocols based on the extracted at least one of gesture, injury, and motion information.

In another aspect, the present disclosure recites one or more processor to process the received input data for optimizing a medical imaging examination of a user. The one or more processor is configured to extract, from the received input data, one or more protocol parameters and features corresponding to the extracted one or more protocol parameters. The one or more processor is further configured to identify the at least one protocol parameter among the plurality of protocol parameters based on the extracted features. The one or more processor is further configured to control the change in the at least one protocol parameter with respect to the extracted one or more protocol parameters, based on at least one of the priority level and suggested change falling within the pre-defined range of the at least one protocol parameters.

In another aspect, the present disclosure recites one or more processor to process the received input data for optimizing a medical imaging examination of a user. The one or more processor is configured to when the priority level is indicated high, change the at least one parameter with respect to the extracted one or more protocol parameters whether the suggested change falls within the pre-defined range or outside the pre-defined range. The one or more processor is further configured to when the priority level is indicated low, control the change in the at least one protocol parameter with respect to the extracted one or more protocol parameters based on the medical information whether the suggested change falls within the pre-defined range or outside the pre-defined range.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The embodiments of the disclosure itself, as well as a preferred mode of use, further objectives and advantages thereof, will best be understood by reference to the following detailed description of an illustrative embodiment when read in conjunction with the accompanying drawings. One or more embodiments are now described, by way of example only, with reference to the accompanying drawings in which:

FIG. 1A illustrates an environment architecture for implementing artificial intelligence (AI) based protocol selection system, in accordance with an embodiment of the present disclosure.

FIG. 1B illustrates by way of a block diagram of an artificial intelligence (AI) based protocol selection system, in accordance with an embodiment of the present disclosure.

FIG. 2 illustrates by way of a block diagram of an AI based protocol selection system for retrieving medical information of the user, in accordance with an embodiment of the present disclosure.

FIG. 3 illustrates by way of a block diagram of an AI based protocol selection system for selecting one or more protocols, in accordance with an embodiment of the present disclosure.

FIGS. 4A-C illustrates by way of block diagrams of an identification unit for identifying protocol parameters, in accordance with an embodiment of the present disclosure.

FIG. 5 illustrates by way of a block diagram of an AI based protocol selection system for retrieving medical information of the user, in accordance with an embodiment of the present disclosure.

FIG. 6 a flow diagram illustrating an AI based protocol selection method, in accordance with an embodiment of the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

The foregoing has broadly outlined the features and technical advantages of the present disclosure in order that the detailed description of the disclosure that follows may be better understood. It should be appreciated by those skilled in the art that the conception and specific embodiment disclosed may be readily utilized as a basis for modifying or designing other structures for carrying out the same purposes of the present disclosure.

Various embodiments of the present invention now will be described more fully hereinafter with reference to the accompanying drawings, in which some, but not all embodiments of the invention are shown. Indeed, the invention may be embodied in many different forms and should not be construed as limited to the embodiments set forth herein. Rather, these embodiments are provided so that this disclosure will satisfy applicable legal requirements. The term “or” is used herein in both the alternative and conjunctive sense, unless otherwise indicated. The terms “illustrative,” “example,” and “exemplary” are used to be examples with no indication of quality level. Like numbers refer to like elements throughout.

The phrases “in an embodiment,” “in one embodiment,” “according to one embodiment,” and the like generally mean that the particular feature, structure, or characteristic following the phrase may be included in at least one embodiment of the present disclosure and may be included in more than one embodiment of the present disclosure (importantly, such phrases do not necessarily refer to the same embodiment).

The word “exemplary” is used herein to mean “serving as an example, instance, or illustration.” Any implementation described herein as “exemplary” is not necessarily to be construed as preferred or advantageous over other implementations.

If the specification states a component or feature “can,” “may,” “could,” “should,” “would,” “preferably,” “possibly,” “typically,” “optionally,” “for example,” “often,” or “might” (or other such language) be included or have a characteristic, that particular component or feature is not required to be included or to have the characteristic. Such component or feature may be optionally included in some embodiments, or it may be excluded.

The phrase “artificial intelligence” refers to the field of studying artificial intelligence or methodology for making artificial intelligence, and machine learning refers to the field of defining various issues dealt with in the field of artificial intelligence and studying methodology for solving the various issues. Machine learning is defined as an algorithm that enhances the performance of a certain task through a steady experience with the certain task.

The phrase “machine learning” is used throughout the disclosure. The machine learning broadly describes a function of systems that learn from data. A machine learning system, engine, or module can include a machine learning algorithm that can be trained to learn functional relationships between inputs and outputs that are currently unknown. In one or more embodiments, machine learning functionality can be implemented using an artificial neural network (ANN) having the capability to be trained to perform a currently unknown function. In machine learning and cognitive science, ANNs are a family of statistical learning models inspired by the biological neural networks of animals, and in particular the brain. ANNs can be used to estimate or approximate systems and functions that depend on a large number of inputs.

Machine learning may be classified into supervised learning, unsupervised learning, and reinforcement learning according to a learning method. The supervised learning may refer to a method of learning an artificial neural network in a state in which a label for learning data is given, and the label may mean the correct answer (or result value) that the artificial neural network must infer when the learning data is input to the artificial neural network. The unsupervised learning may refer to a method of learning an artificial neural network in a state in which a label for learning data is not given. The reinforcement learning may refer to a learning method in which an agent defined in a certain environment learns to select a behavior or a behavior sequence that maximizes cumulative compensation in each state.

Disclosed herein is an artificial intelligence (AI) based protocol selection method and system for optimizing medical imaging examination of a user. The AI based protocol selection system identifies user physical information, wherein the user physical information includes at least one of gesture, height, weight, injury, motion, and structure information of the user. The system simultaneously retrieves medical information of the user. The medical information of the user corresponds to at least one of a medical history of the user and current medical assessment information of the user. The system then selects one or more medical examination protocols based on at least a part of the identified user physical information and identifies a plurality of protocol parameters for at least one medical examination. To identify the a plurality of protocol parameters the system uses the retrieved user medical information, the selected one or more medical examination protocols, and at least a part of the identified user physical information. The system further receives input information tagged with a priority level, wherein the received input information is related to a change in at least one of the protocol parameters from the plurality of protocol parameters. The received input information is then processed to change at least one of protocol parameters from the plurality of protocol parameters based on at least one of the priority level and a pre-defined range of the at least one of protocol parameters from the plurality of protocol parameters. Finally, the system provides the plurality of protocol parameters based on the processed input information to a medical imaging apparatus for performing the medical imaging examination of the user. In this regard, the system performs an automated procedure in order to select examination protocol and in order to identify protocol parameters for medical imaging examination. Thus, the system reduces chances of error in selection of the medical examination protocol. Further, the system minimizes the protocol parameters selection time than the conventional procedure.

Turning now to the drawings, the detailed description set forth below in connection with the appended drawings is intended as a description of various configurations and is not intended to represent the only configurations in which the concepts described herein may be practiced. The detailed description includes specific details for the purpose of providing a thorough understanding of various concepts with like numerals denote like components throughout the several views. However, it will be apparent to those skilled in the art that these concepts may be practiced without these specific details.

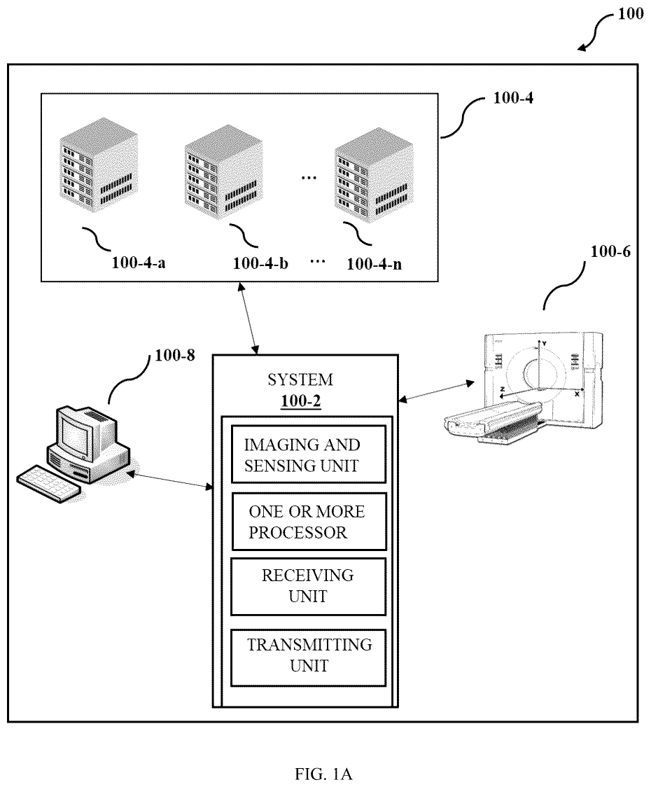

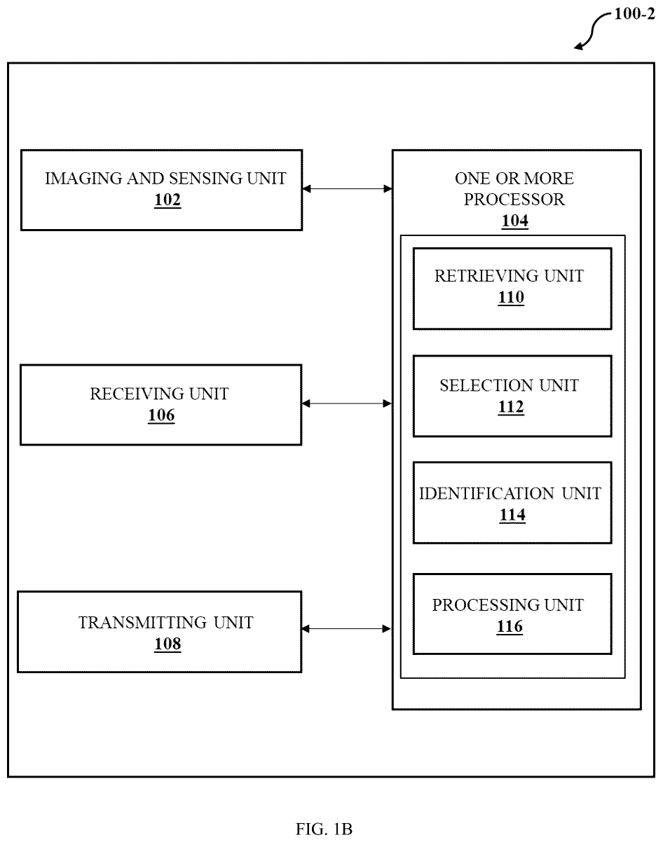

FIG. 1A illustrates an environment architecture for implementing an AI based protocol selection system of the present invention, in accordance with an embodiment of the present disclosure. The environment architecture 100 may be constituted by an AI based protocol selection system 100-2, one or more servers 100-4, an imaging apparatus 100-6, a terminal device 100-8. All the constituent elements of the environment architecture 100 illustrated in FIG. 1A are not essential constituent elements, and the environment architecture 100 may be implemented by more constituent elements than the constituent elements illustrated in FIG. 1A. However the same are not explained for the sake of brevity. All the constituent elements of the environment architecture 100 may communicate with each other via wireless/wired communication network.

In an embodiment, the system 100-2 may be constituted by an imaging and sensing unit, one or more processor, a receiving unit, and a transmitting unit. Further, the constituted elements of the system 100-2 may communicate with all other constituent elements of the environment architecture 100 in order to select medical examination protocol and identify protocol parameters based on the selected medical examination protocol. The detailed functioning of the system 100-2, in conjunction with other element disclosed in FIG. 1A, is further explained in FIG. 1B in forthcoming paragraphs of the present disclosure.

In an embodiment, the one or more servers 100-4 may represent a Hospital Information System (HIS)/Radiology Information System (RIS) server. The HIS/RIS servers 100-4 is a system used in hospitals and medical centers to manage patient/user information and radiology images. The server may communicate with the system 100-2 in order to provide the patient/user information and radiology images. In an embodiment, the one or more servers 100-4 may include n number of servers such as a first server 100-4-a, a second server 100-4-b, and nth server 100-4-n. The system 100-2 may communicate either with one server among the one or more servers 100-4 or more than one server among the one or more servers 100-4.

In particular, the HIS is a comprehensive, integrated system that is used to manage and coordinate all aspects of a hospital's operations, from patient admissions and medical billing to inventory management and resource planning. It includes modules for patient registration, electronic medical records (EMRs), clinical decision support, laboratory and radiology information systems, and pharmacy management. Further, the RIS is a specialized module within a HIS that is used to manage and organize radiology department operations, including scheduling, image capture and storage, report generation, and communication with other healthcare providers. The RIS system can also integrate with Picture Archiving and Communication System (PACS) to provide a complete solution for managing radiology images.

Further, the imaging apparatus 100-6 may perform imaging of a user by using the protocol parameters received from the system 100-2. In an embodiment the imaging apparatus 100-6 may be a part of the system 100-2. In an alternative embodiment the imaging apparatus 100-6 may be an entity different from the system 100-2.

In particular, the imaging apparatus 100-6 for medical imaging is a device or system that is used to create images of the internal structures of the human body for medical diagnosis or treatment. The imaging apparatus 100-6 may be, not limited to, a computed tomography (CT), magnetic resonance imaging (MRI), ultrasound, and so forth. the imaging apparatus 100-6 are an essential tool in modern medicine, as they allow doctors and medical professionals to see inside the body without the need for invasive procedures. By using these devices, medical professionals can detect and diagnose various diseases and conditions, including cancer, heart disease, neurological disorders, and musculoskeletal injuries.

Further, the terminal device 100-8 may be any electronic device, for example, an instant messaging electronic device, a game electronic device, a social platform electronic device, an email electronic device, an audio/video electronic device, a tablet computer, a mobile phone, an electronic reader, a remote control, a personal computer (PC), a notebook computer, a notebook computer, on-board equipment, a network television or a wearable device. A technician/physician may operate the terminal device 100-8 in order to exchange the information with the system 100-2.

In particular, a technician, also known as a medical technologist or clinical laboratory scientist, may be responsible for performing laboratory tests and procedures on patient samples. The technician may analyze and interpret results, and provide accurate and timely information to physicians for diagnosis and treatment. Further, a physician may be a medical professional who is trained to diagnose and treat illnesses and injuries. The Physician may have a much broader scope of responsibility and can work in a variety of settings, such as hospitals, clinics, private practices, or research facilities.

FIG. 1B illustrates an artificial intelligence (AI) based protocol selection system 100-2 for optimizing a medical imaging examination of a user, in accordance with an embodiment of the present disclosure. It must be understood to a person skilled in art that architecture may also be implemented in various environments, other than as shown in FIG. 1.

According to an embodiment of the present disclosure, the AI based protocol selection system 100-2 may be constituted by an imaging and sensing unit 102, one or more processor 104, a receiving unit 106, and a transmitting unit 108. All the constituent elements of the system 100-2 illustrated in FIG. 1B are not essential constituent elements, and the system 100-2 may be implemented by more constituent elements than the constituent elements illustrated in FIG. 1B, however the same are not explained for the sake of brevity. All the constituent elements of the system 100-2 communicate with each other via wireless/wired communication network.

In a non-limiting embodiment of the present disclosure, an imaging and sensing unit 102 may be configured to identify user physical information. The user physical information may include at least one of gesture, height, weight, injury, motion, and structure information of the user.

In particular, the imaging unit and sensing unit 102 may be a multi-modality 3D sensor camera that captures a patient/user gesture, injuries, structure, and motion in three dimensions. These cameras use a combination of different sensing modalities, such as depth sensing, color imaging, and infrared imaging, to provide a detailed and accurate representation of the user body.

A multi-modality 3D sensor camera is a camera device that captures three-dimensional images of the user and scenes by using multiple sensing modalities. The multiple sensing modalities are, not limited to, stereo cameras, structured light, time-of-flight (TOF), active/passive infrared (IR) imaging, and so forth. The multi-modality 3D sensor camera depends on the specific modalities used in the device 100-2. In particular, the multi-modality 3D sensor camera captures two or more images of the user from different angles, using multiple sensing techniques. These captured images of the user are used to identify different physical information of the user.

Further, the imaging and sensing unit 102 may include a sensor to detect the user weight and height. The sensor may be, not limited to, a scale sensor, a load sensor, an ultrasonic sensor, a laser sensor, a camera sensor, and so forth. The imaging and sensing unit 102 may include one of the above-mentioned sensors or combination of the above-mentioned sensors. The imaging and sensing unit 102 may provide the physical information of the user to the selection unit 112. The imaging and sensing unit 102 may capture multiple images or video of patient/user related to different poses of the patient/user in order to capture complete body structure of the patient/user.

In a non-limiting example of the present disclosure, when a physician advised to perform medical examination of the user by using the medical imaging apparatus, then the system 100-2 may be configured to capture multiple images of the user by using the camera. Further, the system 100-2 using the imaging and sensing unit 102, may be configured to identify the weight and height of the user when the user is in contact with the system 100-2.

Further, the system 100-2 may include the one or more processor 104. The one or more processor 104 are constituted by at least a retrieving unit 110, a selection unit 112, an identification unit 114, and a processing unit 116. All the constituent elements included in the one or more processor 104 illustrated in FIG. 1B are not essential constituent elements, and the one or more processor 104 may be implemented by more constituent elements than the constituent elements illustrated in FIG. 1B, however the same are not explained for the sake of brevity. All the constituent elements of the one or more processor 104 communicate with each other via wireless/wired communication network.

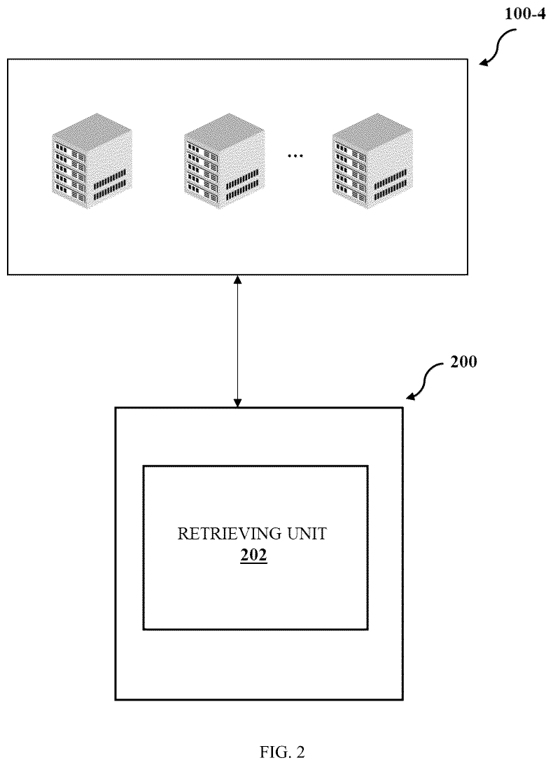

In a non-limiting embodiment of the present disclosure, the retrieving unit 110 may be configured to retrieve medical information of the user, wherein the user medical information corresponds to at least one of a medical history and current medical assessment of the user. The retrieving of the medical information by retrieving unit 110 is further explained in FIG. 2, in forthcoming paragraphs.

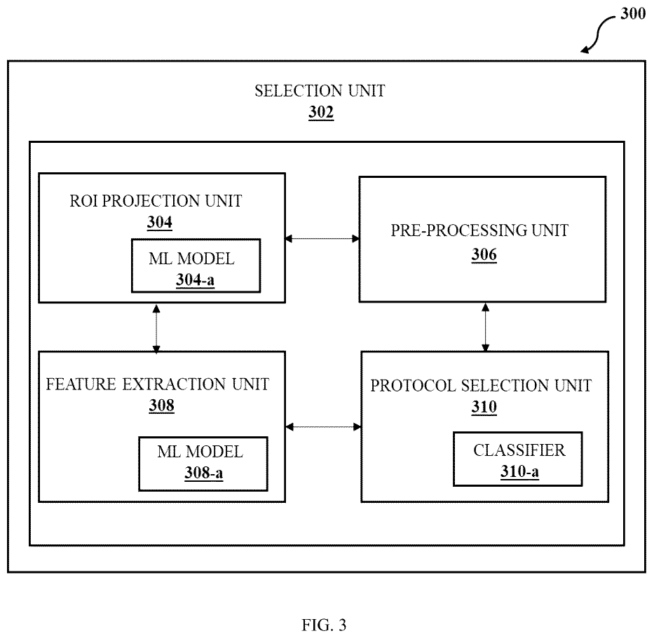

In a non-limiting embodiment of the present disclosure, the selection unit 112 may be configured to select one or more medical examination protocols based on at least a part of the identified user physical information. The selection unit 112 may receive the physical information from the imaging and sensing unit 102 and select the medical examination protocols based on the received physical information. The selection of medical examination protocols by the selection unit 112 is further explained in FIG. 3 in detail in forthcoming paragraphs of the present disclosure. Those skilled in the art will appreciate that the protocol may be, not limited to, Head Computed Tomography (CT), Chest CT, Abdominal CT, Pelvic CT, Angiography CT, Brain Magnetic resonance imaging (MRI), Spine MRI, Abdominal MRI, Pelvic MRI, Angiography MRI, and so forth. The selection unit 112 includes a machine learning (ML) model that selects the one or more medical examination protocols.

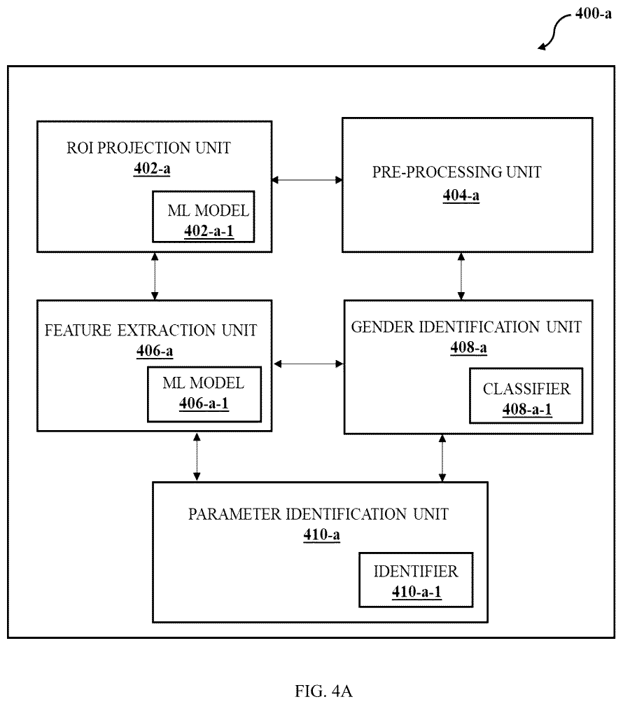

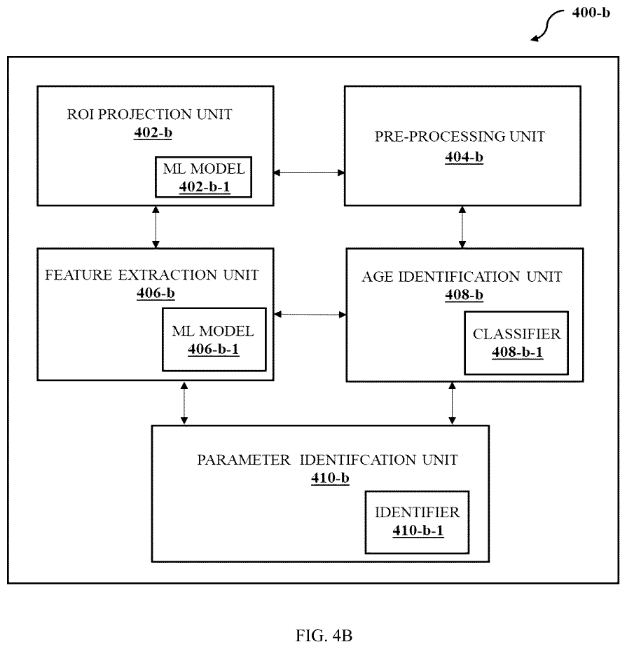

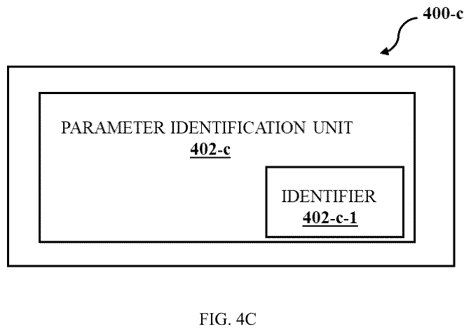

In a non-limiting embodiment of the present disclosure, an identification unit 114 may be configured to identify a plurality of protocol parameters for the medical examination based on the retrieved user medical information and the selected one or more medical examination protocols. The plurality protocol parameters are related to at least one of, not limited to, scan mode, slice thickness, scan time, contrast injection, reconstruction algorithm, field of view, matrix size, echo time, repetition time, flip angle, and so forth. The identification unit 114 may be configured to receive the medical information from the retrieving unit 110 and the medical examination protocol selection information from the selection unit 112. Further, the identification unit 114 may be configured to identify the plurality of protocol parameters for the medical examination by using the received medical information and the selected medical examination protocol information. The identification of the plurality of protocol parameters is further explained in FIG. 4 in detail in the forthcoming paragraphs of the present disclosure. It is to be appreciated that the identification unit 114 may include an ML model that identifies the plurality of protocol parameters.

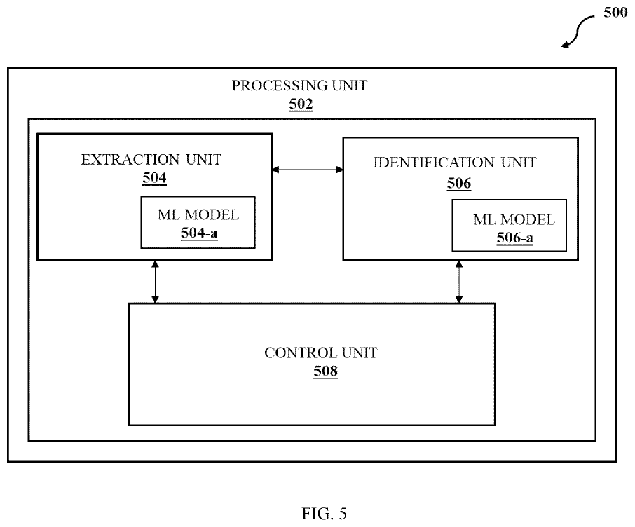

In a non-limiting embodiment of the present disclosure, the processing unit 116 may be configured to process an input data that is received from the receiving unit 106 to change the at least one protocol parameter based on at least one of a priority level and a pre-defined range of the at least one protocol parameter. In particular, the processing unit 116 includes an ML model that performs processing whether to change any protocol parameter among the plurality of protocol parameters. The processing unit 116 first provides the identified protocol parameters to the technician or physician via using the transmitting unit 108. The physician or technician checks the protocol parameters and provides input data regarding change of one or more protocol parameters among the plurality of parameters. The processing unit 116 may then process the input data in order to check whether the change of one or more protocol parameters is required or not. If the change is required, then the processing unit 116 changes the one or more protocol parameters. The processing of the input data related to change of the one or more protocol parameters is further explained in FIG. 5, in detail in forthcoming paragraphs of the present disclosure.

In a non-limiting embodiment of the present disclosure, the receiving unit 106 is communicatively coupled to the one or more processor 104. The receiving unit 106 may be configured to receive the input data tagged with the priority level. The received input data is related to a change in at least one protocol parameter among a plurality of protocol parameters. In particular, after the identification of the protocol parameters, the technician or physician provides the input data that includes new parameters information, an instruction to change one or more parameters, and priority level of the instruction. The receiving unit 106 provides the input data to the processing unit 116.

The receiving unit 106 may be, for example, a receiver that may include an antenna, an antenna array, an input interface, a pin, a circuit, or the like. The receiving unit 106 may receive input data either through the external device or through the manual input provided in the system 100-2. The input received through the external device may be not limited to voice data, image data, and so forth.

In a non-limiting embodiment of the present disclosure, the transmitting unit 108 operatively coupled to the one or more processor 104 and may be configured to provide the plurality of protocol parameters based on the processed received input data to a medical imaging apparatus for performing the medical imaging examination of the user. In particular, the transmitting unit 108 may receive the identified protocol parameters from the identification unit 114 and provide the identified protocol parameters to the technician or physician. Further, the transmitting unit 108 may receive the protocol parameters from the processing unit 116 after the processing of the input data by the processing unit 116. The transmitting unit 108 may provide the protocol parameters to the medical imaging apparatus. The transmitting unit 108 may be, for example, a transmitter that includes an antenna or antenna array, an output interface, a pin, a circuit, or the like. The transmitting unit 108 may provide data to the medical imaging apparatus. Thus, the system 100-2 performs an automated procedure in order to select medical examination protocol and in order to identify protocol parameters for medical imaging examination. The system 100-2 reduces chances of error in selection the medical examination protocol. Further, the system 100-2 minimizes the protocol parameters selection time than the conventional procedure.

FIG. 2 illustrates an AI based protocol selection system 200 (same as the protocol selection system 100-2 of FIG. 1B) for retrieving medical information of the user. The system 200 may include a retrieving unit 202 (same as the retrieving unit 110 of FIG. 1B) for retrieving medical information of the user. The system 200 may be in communication connection with HIS/RIS server (such as the HIS/RIS 100-4 of FIG. 1A).

In a non-limiting example of the present embodiment, when the user comes to the hospital during an medical emergency, the physician will perform an examination of the user and may suggest a medical imaging procedure based on the examination. Before the medical imaging procedure, the physician will advise the user to undergo some medical tests/assessments. The medical assessment may be, not limited to, blood test, urine test, physical examination, pulmonary function test, and so forth. This medical assessment provides information about current medical information of the user and the medical history of the user. The physician provides the current medical information of the user and the medical history of the user may be available/retrieved from the HIS/RIS server 100-4, based on the Medical ID associated with the user. Precisely, the HIS/RIS server 100-4 may store the current medical information of the user and the medical history of the user which is required for medical examination of the user. Those skilled in the art will appreciate that the HIS/RIS server 100-4 may be a database used in hospitals and medical centers to manage patient/user information and radiology images.

Now, as shown in FIG. 2, the retrieving unit 204 may be communicably coupled with the HIS/RIS server 100-4. Thus, the retrieving unit 204 may be configured to retrieve the medical information of the user from the HIS/RIS server 100-4. The medical information may include current medical information which is called as current medical assessment of the user. Further, the medical information may include the medical history of the user.

In an alternative embodiment of the present disclosure, some hospital may not have facility of the HIS/RIS server 100-4. In this scenario, the physician or technician unable to the store the assessment information in the HIS/RIS server 100-4. The physician or technician may directly provide the assessment information to a storage (not shown in FIG. 2) of the system 200. The storage is different from the HIS/RIS server 100-4. The retrieving unit 204 may extract the medical information of the user directly from the storage of the system 200.

Moving on to FIG. 3 that illustrates an AI based protocol selection system 300 (same as the protocol selection unit 100-2 of FIG. 1B) for selecting one or more medical examination protocols. As discussed in forgoing paragraphs in view of description of FIG. 1B, the protocol selection system 300 may include a selection unit 302 (same as the selection unit 112 of FIG. 1B) that may be configured to select one or more medical examination protocols based on the user physical information by using multiple constituent elements. The constituent elements of the selection unit 302 may include one or more sub-units including but not limited to a region of interest (ROI) projection unit 304, a pre-processing unit 306, a feature extraction unit 308, and a protocol selection unit 310. All the constituent elements of the system 300 illustrated in FIG. 3 are not essential constituent elements, and the system 300 may be implemented by more constituent elements than the constituent elements illustrated in FIG. 3, however the same are not explained for the sake of brevity. All the constituent elements of the system 300 communicate with each other via wireless/wired communication network.

In a non-limiting embodiment of the present disclosure, the selection unit 302 may be configured to perform region of interest projection on the identified user physical information, wherein the identified user physical information corresponds to imaging data, received in the form of an image or a video, of the user. Further, the selection unit 302 may be configured to extract the at least one of gesture, injury, and motion information from the identified user physical information based on the region of interest projection and select the one or more medical examination protocols based on the extracted at least one of gesture, injury, and motion information.

The ROI projection unit 304 may provide a technique to extract a specific area of interest from an image or video stream. The ROI projection unit 304 may receive a plurality of images or video from the imaging and the sensing unit (such as the imaging and sensing unit 102). The plurality of images or video may indicate the user physical information. The user physical information may include at least one of gesture, injury, motion, and structure information of the user.

After receiving the physical information, the ROI projection unit 304 may identify multiple regions of interest in the plurality of images or video. The region of interest is identified by a ML model 304-a present in the ROI projection unit 304. In an embodiment, the ML model 304-a is trained to perform gesture recognition, injury recognition, and motion recognition from the plurality of images or video. The ML model that is associated with ROI projection unit 304 may be, not limited to, an object detection model, a segmentation model, a classification model, a regression model, a deep learning model, and so forth. The ROI projection unit 304 may be configured to extract relevant pixels of the identified multiple regions of interest. The ROI projection unit 304 may project the extracted relevant pixels onto a new images or video frame using a transformation matrix that maps the regions of interest coordinates to the new images or video frame. The new images or video can be written as ROI images or video.

Further, the pre-processing unit 306 may be configured to perform a processing to extract unwanted information from the ROI images or video. In particular, the pre-processing unit 306 first identifies unwanted regions from the ROI images or video. The unwanted regions may be, not limited to, noise, artifacts, irrelevant features, and so forth. The pre-processing unit 306 may then remove the extracted unwanted information from the ROI images by using different techniques. The techniques may be, not limited to, a thresholding technique, a filtering technique, a segmentation technique, or any other similar technique known in the art.

In an another essential aspect, the feature extraction unit 308 may be configured to receive the images or video from the pre-processing unit 306. The features extraction unit 308 may include another ML model 308-a that is used to identify the features from the images or video. The ML model 308-a related to features extraction unit 308 may be, not limited to, a conventional neural network, a support vector machine, a random forest, a deep belief network, an autoencoder, and so forth. The ML model 308-a related to features extraction unit 308 may employ one or more known features extraction technique in order to extract the features from the images or video. The extracted features may be, not limited to, shape information, texture information, color information, and intensity information from the images or video. For example, the ML model 308-a related to features extraction 308 is trained to identify features related to bloodshot eye injury. The feature extraction unit 308 first receives the images or video and identify the bloodshot eye in the received images or video. After the reception, the feature extraction unit 308 extracts the features related to bloodshot eye.

In a specific embodiment, the protocol selection unit 310 may include an ML model based trained protocol classifier 310-a. The trained protocol classifier 310-a may be trained on a dataset of images that provides protocol information associated with injury in the images or video based on the extracted features. The trained protocol classifier may be, not limited to, a decision three classifier, a random forest classifier, a support vector machine classifier, a neural network classifier, and so forth. The protocol selection unit 310 may receive the extracted parameter from the features extraction unit 308 and select one or more medical examination protocols by using the extracted features.

Referring now to FIG. 4A-4C that illustrates an identification unit 400 (same as the identification unit 114 of FIG. 1B) of a protocol selection system (such as the protocol selection system 100-2 of FIG. 1B) for identifying protocol parameters. The identification unit 400 is constituted by a first identification unit 400-a, a second identification unit 400-b, and a third identification unit 400-c.

As shown in FIG. 4A, the first identification unit 400-a may include but not limited to a ROI projection unit 402-a, a pre-processing unit 404-a, a feature extraction unit 406-a, a gender identification unit 408-a, and a parameter identification unit 410-a. All the constituent elements 402-a, 404-a, 406-a, 408-a, and 410-a of the system 400-a illustrated in FIG. 4A are not essential constituent elements, and the first identification unit 400-a may be implemented by more constituent elements than the constituent elements illustrated in FIG. 4A, however the same are not explained for the sake of brevity. All the constituent elements of the first identification unit 400-a communicate with each other via wireless/wired communication network.

The ROI projection unit 402-a may be configured to extract a specific area of interest from an image or video stream. The ROI projection unit 402-a receives a plurality of images or video from the imaging and the sensing unit (such as the imaging and sensing unit 102 of FIG. 1B). The plurality of images or video indicates a structure information of the user.

After receiving the structural information of the user, the ROI projection unit 402-a identifies multiple regions of interest in the plurality of images or video. The region of interest is identified by an ML model 402-a-1 present in the projection unit 402-a. The ML model 402-a-1 related to the projection unit 402-a is trained to perform gender recognition from the plurality of images or video. The ML model 402-a-1 related to the projection unit 402-a is, not limited to, a conventional neural network, a support vector machine, a K-nearest neighbor, a naïve bayes, and so forth. To identify the gender information from images or video, ROI may be, not limited to, a face ROI, a clothing ROI, a hair ROI, and so forth but various other parameters that may be important to differentiate genders based on aesthetic appearance. To achieve this, the ROI projection unit 402-a may extract relevant pixels of the identified multiple regions of interest. The ROI projection unit 402-a may then project the extracted relevant pixels onto a new images or video frame using a transformation matrix that maps the regions of interest coordinates to the new images or video frame. The new images or video can be written as ROI images or video.

Further, the pre-processing unit 404-a may be configured to perform processing, to extract unwanted information from the ROI images or video. In particular, the pre-processing unit 404-a may first identify unwanted regions from the ROI images or video. The unwanted regions may be, not limited to, noise, artifacts, irrelevant features, and so forth but may include other unwanted features/information. The pre-processing unit 404-a may remove the extracted unwanted information from by using different techniques. The techniques may be, not limited to, a thresholding technique, a filtering technique, a segmentation technique, and so forth.

Simultaneously, the feature extraction unit 406-a may be configured to receive the images or video from the pre-processing unit 404-a. The features extraction unit 406-a may include an ML model 406-a-1 that is used to identify the features from the images or video. The ML model 406-a-1 related to features extraction unit 406-a may be, not limited to, a conventional neural network, a support vector machine, a random forest, a deep belief network, an autoencoder, and so forth. The ML model 406-a-1 related to features extraction unit 406-a may include one or more known features extraction techniques that may be used to extract the features from the images or video. The extracted features may be, not limited to, a colour, a texture, a shape, hair, facial, and so forth.

Further, the gender identification unit 408-a includes an ML model based trained gender classifier 408-a-1. In an embodiment, the trained gender classifier 408-a-1 for gender classification is trained on a dataset of images that provides gender information associated in the images or video based on the extracted features. Those skilled in the art will appreciate that the trained gender classifier 408-a-1 may be, not limited to, conventional neural networks, support vector machines, a decision tree, a random forest, naïve bayes, and so forth. In an embodiment, the gender identification unit 408-a may receive the user height and weight information from the imaging and sensing unit 102. Alternatively, the technical or physician (not shown) may provide the user weight and height information to the system 400-a. Further, the gender identification unit 408-a may receive the extracted features from the feature extraction unit 406-a. After receiving both the information, the gender identification unit 408-a may identify that the user present in the images or video is either of a male or female or transgender. The first identification unit 400-a may provide correct identification of the gender than the conventional gender identification method which enables efficient medical examination of the user.

In a non-limiting example of present disclosure, the first identification unit 400-a may perform ROI projection on an image and extract face ROI from the image. Further, the first identification unit 400-a may extract facial information (i.e. mustache information) from the extracted face ROI. The first identification unit 400-a may identify that the person is male based on the extracted facial information.

Furthermore, the parameter identification unit 410-a may include an ML model based trained parameter identifier 410-a-1 that provides protocol parameters associated with the selected one or more medical examination protocols by using the gender information. The trained parameter identifier 410-a-1 may be, not limited to, a logistic regression, a neural network, a support vector machine, and so forth. The parameter identification unit 410-a may be configured to receive the protocol information from the selection unit (such as the selection unit 112 of FIG. 1B). The protocol information may include one or more medical examination protocols that are selected by the selection unit (such as the selection unit 112 of FIG. 1B) using the user physical information. The parameter identification unit 410-a may identify the protocol parameters for each of the selected one or more medical examination protocols by using the gender information.

As shown in FIG. 4B, the second identification unit 400-b may include but not be limited to include a ROI projection unit 402-b, a pre-processing unit 404-b, a feature extraction unit 406-b, an age identification unit 408-b, and a parameter identification unit 410-b. The constituent elements 402-b, 404-b, 406-b, 408-b, and 410-b of the second identification unit 400-b illustrated in FIG. 4B are not essential constituent elements, and the system 400-b may be implemented by more constituent elements than the constituent elements illustrated in FIG. 4B, however the same are not explained for the sake of brevity. All the constituent elements of the second identification unit 400-b communicate with each other via wireless/wired communication network.

In an embodiment, the ROI projection unit 402-b may be configured to extract a specific area of interest from an image or video stream, received from the imaging and the sensing unit 102. In a specific embodiment, the ROI projection unit 402-b may be configured to receive a plurality of images or video from the imaging and the sensing unit (such as the imaging and sensing unit 102 of FIG. 1B). The plurality of images or video indicates a structure information of the user.

After receiving the structural information of the user, the ROI projection unit 402-b may identify multiple regions of interest in the plurality of images or video. The region of interest may be identified using an ML model 402-b-1 present in the ROI projection unit 402-b. The ML model 402-b-1 related to ROI projection unit 402-b may be a trained ML model 402-b-1 configured to perform gender recognition from the plurality of images or video. In an aspect, the ML model 402-b-1 of the ROI projection unit 402-bmay be, a deep neural network, a random forest, a support vector machine, a conventional neural network, or any other similar model. To identify age information from images or video, ROI may be, not limited to, a face ROI, a bone ROI, a skin ROI, and so forth. The ROI projection unit 402-b may extract relevant pixels of the identified multiple regions of interest. The ROI projection unit 402-b may project the extracted relevant pixels onto a new images or video frame using a transformation matrix that maps the regions of interest coordinates to the new images or video frame. The new images or video can be written as ROI images or video.

Moving on, the pre-processing unit 404-b of FIG. 4b, may be configured to process/extract unwanted information from the ROI images or video. In particular, the pre-processing unit 404-b may first identify unwanted regions from the ROI images or video. The unwanted regions may be, not limited to, noise, artifacts, irrelevant features, and so forth. The pre-processing unit 404-b may then remove the extracted unwanted information from ROI images or video by using different techniques. These techniques may be, not limited to, a thresholding technique, a filtering technique, a segmentation technique, and so forth.

The feature extraction unit 406-b received the images or video from the pre-processing unit 404-b. The features extraction unit 406-b includes an ML model 406-b-1 that is used to identify the features from the images or video. The ML model 406-b-1 related to features extraction unit 406-b may be, not limited to, a conventional neural network, a support vector machine, a random forest, a deep belief network, an autoencoder, and so forth. The ML model 406-b-1 related to features extraction unit 406-b may include a features extraction technique in order to extract the features from the images or video. The extracted features may be, not limited to, wrinkle depth and density, skin texture and roughness, joint space width, bone density, and so forth.

The age identification unit 408-b includes an ML model based trained age classifier 408-b-1. The trained age classifier 408-b-1 is trained on a dataset of images that provides age information associated in the images or video based on the extracted features. The trained age classifier 408-b-1 may be, not limited to, conventional neural networks, support vector machines, random forest, gradient boosting, and so forth. The age identification unit 408-b receives the user height and weight information from the imaging and sensing unit 102. Alternatively, the technical or physician manually provides the user weight and height information to the second identification unit 400-b. Further, the age identification unit 408-b may receive the extracted features from the feature extraction unit 406-b. After receiving both the information, the age identification unit 408-b may identify that the person present in the images or video is one of a child, adult, or old age. Further, the age identification unit 408-b may identify the age of the person within a specific range. The first identification unit 400-a may provide correct identification of the age than the conventional gender identification method which enables efficient medical imaging examination of the user.

In a non-limiting example of present disclosure, the second identification unit 400-b performs ROI projection on an image and extract skin ROI from the image. Further, the second identification unit 400-b may extract skin information facial information (i.e. skin texture and roughness) from the extracted face ROI. The second identification unit 400-b may identify that the person is an adult, and the age of the person is between 30-35.

The parameter identification unit 410-b includes an ML model based trained parameter identifier 410-b-1 that provides protocol parameters associated with the selected one more medical examination protocols by using the age information. The trained parameter identifier 410-B-1 may be, not limited to, a logistic regression, a neural network, a support vector machine, a decision tree, and so forth. The parameter identification unit 410-b may receive the protocol information from the selection unit (such as the selection unit 112 of FIG. 1B). The protocol information may include one or more medical examination protocols that are selected by the selection unit 112 using the physical information. The parameter identification identifies the protocol parameters for each of the selected one or more medical examination protocols by using the age information.

In view of FIG. 4C, the third identification unit 400-c includes a parameter identification unit 402-c. The parameter identification unit 402-c includes ML based trained parameter identifier 402-c-1 that provides protocol parameters associated with the selected one or more medical examination protocols based on the medical information. The trained parameter identifier may be, not limited to, decision trees, random forests, a support vector machine, a neural network, and so forth. The parameter identification unit 402-c may receive the protocol information from the selection unit (such as the selection unit 112 of FIG. 1B). The protocol information may include one or more medical examination protocols that are selected by the selection unit 112 using the physical information. Further, the parameter identification unit 402-c may receive medical information from the retrieving unit (such as the retrieving unit 110 of FIG. 1B). The parameter identification unit 402-c may identify the protocol parameters for each of the selected one or more medical examination protocols by using the medical information. For example, if a protocol i.e. CT scan of the chest is selected and medical information of the user indicates lung disease. Then, the parameter identification unit 402-c may select the parameters that indicates high-resolution CT scan.

Moving on to FIG. 5 that illustrates an AI based protocol selection system 500 (same as the protocol selection system 100-2 of FIG. 1B) for processing of the input data related to change of one or more protocol parameters. The system 500 may include a processing unit 502 (same as the processing unit 116 of FIG. 1B) that changes protocol parameters based on an input data. The input data is received from the terminal device (such as the terminal device 100-8 of FIG. 1A). The constituent elements of the processing unit 502 may include one or more sub-units including but not limited to an extraction unit 504, an identification unit 506, and a control unit 508. All the constituent elements of the system 500 illustrated in FIG. 5 are not essential constituent elements, and the system 500 may be implemented by more constituent elements than the constituent elements illustrated in FIG. 5, however the same are not explained for the sake of brevity. All the constituent elements of the system 500 communicate with each other via wireless/wired communication network.

In a non-limiting embodiment of the present disclosure, the processing unit 502 may be configured to extract, from the received input data, one or more protocol parameters and features corresponding to the extracted one or more protocol parameters. Further, processing unit 502 may be configured to identify the at least one protocol parameter among the plurality of protocol parameters based on the extracted features. Further, the processing unit 502 may be configured to control the change in the at least one protocol parameter with respect to the extracted one or more protocol parameters, based on the priority level and suggested change falling within the pre-defined range of the at least one protocol parameters.

Further, in a non-limiting embodiment of the present disclosure, the processing unit 502 may be configured to, when the priority level is indicated high, change the at least one parameter with respect to the extracted one or more protocol parameters whether the suggested change falls within the pre-defined range or outside the pre-defined range. Further, the processing unit 502 may be configured to, when the priority level is indicated low, control the change in the at least one protocol parameter with respect to the extracted one or more protocol parameters based on the medical information whether the suggested change falls within the pre-defined range or outside the pre-defined range/normal range.

The extraction unit 504 may receive the input data from the receiving unit (such as the receiving unit 106 of FIG. 1B). The input data may include an instruction to change one or more protocol parameters, information of one or more new protocol parameters, and a priority level of the instruction. The priority level of the instruction may indicate the importance of the instruction in order to change the protocol parameters. For example, the high importance level of the instruction may indicate that the technician provides an instruction to change the protocol parameters with the approval of the physician or the physician directly provide the instruction. The low importance level of the instruction indicates that the technician provides an instruction to change the protocol parameters without the approval of the physician. After receiving the input data from the receiving unit 106, the extraction unit 504 may extract features associated with the one or more new protocol parameters.

In a non-limiting example of the embodiment, the input data is in form of image data. The extraction unit 504 may include an ML model 504-a that may perform image processing on the input data and extract the information present in the input data. Further, the ML model 504-a related to the extraction unit 504 may extract the features from the extracted information by a using feature extraction technique such as statistical analysis techniques or machine learning algorithms. The ML model 504-a related to extraction unit 504 may be, not limited to, a conventional neural network, a recurrent neural network, autoencoders, a generative adversarial network, a support vector machine, and so forth.

In a non-limiting example of the embodiment, the input data is in form of voice data. The extraction unit 504 may perform speech recognition or natural language processing techniques to analyze the voice data and extract information associated with the input data. The extraction unit 504 may extract the features from the extracted information by a using feature extraction technique such as statistical analysis techniques or machine learning algorithms. In view of the above examples, the input data is not limited to only voice data or image data. The input data may be manually input data provided by the technician/physician.

Firstly, the identification unit 506 may receive the protocol parameters from each of the first identification unit (such as the first identification unit 402-a of FIG. 4), the second identification unit (such as the first identification unit 402-b of FIG. 4), and the third identification unit (such as the first identification unit 402-c of FIG. 4). After receiving the protocol parameters, the identification unit 506 may consolidate the protocol parameters received from the first identification unit 402-a, the second identification unit 402-b, and the third identification unit 402-c. For example, the identification unit 506 may receive the protocol parameters related to age, protocol parameters related to gender, and protocol parameters related to medical assessment. After receiving the information, the identification unit 506 may consolidate the protocol parameters related to age, gender, and medical assessment by using machine learning technique. After the consolidation, the identification unit 506 may prepare a data set by using the consolidated protocol parameters. The data set may include the consolidated protocol parameters and features associated with the consolidated parameters.

Secondly, the identification unit 506 may receive the input data and compare the features present in the input data with the features present in the data set. After the comparison, the identification unit 506 may identifies features from the data set similar to the features present in the input data. After the identification of the features, the identification unit 506 may extract the protocol parameters from the consolidated protocol parameters. The extracted protocol parameters may related to the identified features. The identification unit 506 may provide the extracted protocol parameters along with the input data to the control unit 508. The identification unit 506 may include an ML model 506-athat may perform consolidation of protocol parameters along with corresponding features and performs comparison functioning. The ML model 506-a related to the identification unit 506 may be, a decision tree, a random forest, a support vector machine, a neural network, a Bayesian network, and so forth.

The control unit 508 may receive the input data and the extracted protocol parameters information from the identification unit 506. The control unit 508 may consider the received information as well as the range of the extracted protocol parameters in to order change the extracted protocol parameters. Description of Table 1, as shown below, indicates the different conditions in which the control unit 508 controls changing of the extracted protocol parameters.

In view of the table, the control unit 508 may check the priority level of the instruction that indicates the change of the extracted protocol parameters among the consolidated protocol parameters by using the protocol parameters indicated in the input data. If the priority level is high, then the control unit 508 may not consider a condition whether the protocol parameters indicated in the input data are within the range of extracted protocol parameters or not. After identifying the high priority level, the control unit 508 may change the extracted protocol parameters with the protocol parameters related to the input data. Once the extracted protocol parameters have been changed, the consolidated protocol parameters are updated. The control unit 508 may provide the updated consolidated protocol parameters to the imaging apparatus (such as the imaging apparatus 100-6 of FIG. 1A) or to a terminal device (such as the terminal device 100-8 of FIG. 1A) by using the transmitting unit (such as the transmitting unit 108 of FIG. 1B).

For example, the system 500 may identify a protocol parameter to decrease the insulin level (x) of the patient, based on consolidated protocol parameters. The normal range of insulin levels for patients/users is between “a” and “b”. The system 500 may receive an input to increase the patient insulin level to a certain level, which has been given high priority. The control unit 508 may check whether the increased insulin level is within the normal range. Since the priority level is high, the control unit 508 may perform increasing of the insulin level regardless of whether the increased insulin level is within the normal range or outside the normal range. Thus, the control unit 508 may adjust the insulin level based on the input received from the technician.

In another exemplary embodiment, if the priority level is low then the control unit 508 may not change the extracted protocol parameters whether the protocol parameters indicated in the input data are within the range of extracted protocol parameters or not. After determination that the parameters are still same, the control unit 508 may provide an error message or a warning message to the imaging apparatus 100-6 or to the terminal device 100-8 by using the transmitting unit 108. The message indicates failure of changing the extracted protocol parameters.

For example, the system 500 may identify a protocol parameter to decrease the insulin level x of the patient consolidated protocol parameters. The normal range of the insulin level for normal patient/user is between “a” and “b”. The system 500 may receive the input to increase the insulin level of the patient at a certain level, which has been given low priority. The control unit 508 may check whether the increased insulin level is within the normal range. Since the priority level is low, the control unit may not perform increasing of the insulin level regardless of whether the increased insulin level is within the normal range or outside the normal range. Therefore, the control unit 508 may not change the protocol parameter to increase the insulin level x of the patient.

TABLE 1

| | Priority | Input data changes fall | Changing of protocol |
|---|---|---|---|
| S. No. | Level | within the range | parameters |
| 1 | High | Yes/No | Based on the input data |
| 2 | Low | Yes/No | No change |

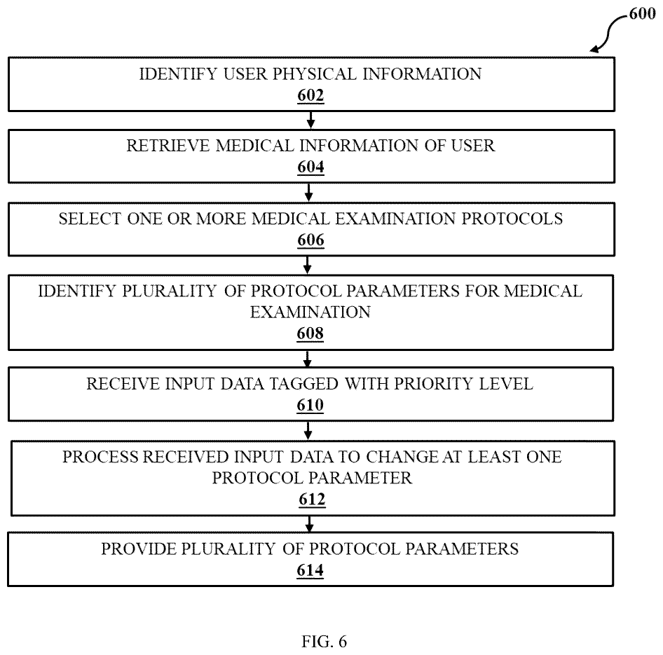

FIG. 6 is a flowchart showing steps of an artificial intelligence (AI) based protocol selection method 600 for optimizing medical imaging examination of a user performed by a system (such as system 100-2 of FIG. 1B). The method starts at step 602, at step 602-the method 600 includes identifying user physical information, wherein the user physical information includes at least one of gesture, height, weight, injury, motion, and structure information of the user. At a step 604, the method 600 includes retrieving medical information of the user, wherein the medical information of the user corresponds to at least one of a medical history and current medical assessment of the user.

At a step 606, the method 600 includes selecting one or more medical examination protocols based on at least a part of the identified user physical information. The selection of the one or more medical examination protocols includes performing region of interest projection on the identified user physical information, wherein the identified user physical information corresponds to imaging data, received in the form of an image or a video, of the user, extracting the at least one of gesture, injury, and motion information from the identified user physical information based on the region of interest projection, and selecting the one or more medical examination protocols based on the extracted at least one of gesture, injury, and motion information.

At a step 608, the method 600 includes identifying a plurality of protocol parameters for the medical examination, based on the retrieved user medical information and the selected one or more medical examination protocols. The identification of the plurality of protocol parameters includes performing region of interest projection on the identified user physical information, wherein the identified user physical information corresponds to imaging data, received in form of an image or a video, of the user, identifying region of interest based on the region of interest projection, determining demographic information of the user based on the identified region of interest, and identifying the plurality of protocol parameters for the medical examination based on the retrieved medical information and the determined demographic information.

At a step 610, the method 600 includes receiving input data tagged with a priority level, wherein the received input data is related to a change in at least one protocol parameter among the plurality of protocol parameters.

At a step 612, the method 600 includes processing the received input data to change the at least one protocol parameter based on at least one of the priority level and a pre-defined range of the at least one protocol parameter. The processing of the received input data includes extracting, from the received input data, one or more protocol parameters and features corresponding to the one or more protocol parameters, identifying the at least one protocol parameter among the plurality of protocol parameters based on the extracted features, and controlling the change in the at least one protocol parameter with respect to the extracted one or more protocol parameters, based on at least one of the priority level and suggested change falls within the pre-defined range of the at least one protocol parameter. The processing of the received input data further includes when the priority level is indicated high, changing the at least one protocol parameter with respect to the extracted one or more protocol parameters whether the suggested change falls within the pre-defined range or outside the pre-defined range, and when the priority level is indicated low, controlling the change in the at least one protocol parameter with respect to the extracted one or more protocol parameters based on the medical information.

At a step 614, the method includes providing the plurality of protocol parameters based on the processed received input data to a medical imaging apparatus for performing the medical imaging examination of the user.

The AI based protocol selection method 600 performing an automated procedure in order to select examination protocol and in order to identify protocol parameters for medical imaging examination. The method 600 reducing chances of error in selecting the medical examination protocol. Further, the method 600 minimizing the protocol parameters selection time than the conventional procedure.

The foregoing method descriptions and the process flow diagrams are provided merely as illustrative examples and are not intended to require or imply that the steps of the various embodiments must be performed in the order presented. As will be appreciated by one of skill in the art the order of steps in the foregoing embodiments may be performed in any order. Words such as “thereafter,” “then,” “next,” etc. are not intended to limit the order of the steps; these words are simply used to guide the reader through the description of the methods. Further, any reference to claim elements in the singular, for example, using the articles “a,” “an” or “the” is not to be construed as limiting the element to the singular.

As used herein, the term unit may be implemented in hardware and/or in software. If the unit is implemented in hardware, the unit may be configured as a device, e.g., as a computer or as a processor or as a part of a system, e.g., a computer system. If the unit is implemented in software, the unit may be configured as a computer program product, as a function, as a routine, or as a program code.

The hardware used to implement the various illustrative logics, logical blocks, modules, and circuits described in connection with the aspects disclosed herein may include a general purpose processor, a digital signal processor (DSP), a special-purpose processor such as an application specific integrated circuit (ASIC) or a field programmable gate array (FPGA), a programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively or additionally, some steps or methods may be performed by circuitry that is specific to a given function.

In one or more example embodiments, the functions described herein may be implemented by special-purpose hardware or a combination of hardware programmed by firmware or other software. In implementations relying on firmware or other software, the functions may be performed as a result of execution of one or more instructions stored on one or more non-transitory computer-readable media and/or one or more non-transitory processor-readable media. These instructions may be embodied by one or more processor-executable software modules that reside on the one or more non-transitory computer-readable or processor-readable storage media. Non-transitory computer-readable or processor-readable storage media may in this regard comprise any storage media that may be accessed by a computer or a processor. By way of example but not limitation, such non-transitory computer-readable or processor-readable media may include random access memory (RAM), read-only memory (ROM), electrically erasable programmable read-only memory (EEPROM), FLASH memory, disk storage, magnetic storage devices, or the like. Disk storage, as used herein, includes compact disc (CD), laser disc, optical disc, digital versatile disc (DVD), floppy disk, and Blu-ray disc™, or other storage devices that store data magnetically or optically with lasers. Combinations of the above types of media are also included within the scope of the terms non-transitory computer-readable and processor-readable media. Additionally, any combination of instructions stored on the one or more non-transitory processor-readable or computer-readable media may be referred to herein as a computer program product.

Many modifications and other embodiments of the inventions set forth herein will come to mind to one skilled in the art to which these inventions pertain having the benefit of teachings presented in the foregoing descriptions and the associated drawings. Although the figures only show certain components of the apparatus and systems described herein, it is understood that various other components may be used in conjunction with the supply management system. Therefore, it is to be understood that the inventions are not to be limited to the specific embodiments disclosed and that modifications and other embodiments are intended to be included within the scope of the appended claims. Moreover, the steps in the method described above may not necessarily occur in the order depicted in the accompanying diagrams, and in some cases one or more of the steps depicted may occur substantially simultaneously, or additional steps may be involved. Although specific terms are employed herein, they are used in a generic and descriptive sense only and not for purposes of limitation.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

What is claimed is:

1. An artificial intelligence (AI) based protocol selection method for optimizing medical imaging examination of a user, comprising:
identifying user physical information, wherein the user physical information includes at least one of gesture, height, weight, injury, motion, and structure information of the user;
retrieving medical information of the user, wherein the medical information of the user corresponds to at least one of a medical history and current medical assessment of the user;
selecting one or more medical examination protocols based on at least a part of the identified user physical information;
identifying a plurality of protocol parameters for the medical examination, based on the retrieved user medical information and the selected one or more medical examination protocols;
receiving input data tagged with a priority level, wherein the received input data is related to a change in at least one protocol parameter among the plurality of protocol parameters;
processing the received input data to change the at least one protocol parameter based on at least one of the priority level and a pre-defined range of the at least one protocol parameter; and
providing the plurality of protocol parameters based on the processed input data to a medical imaging apparatus for performing the medical imaging examination of the user.

2. The method of claim 1, further comprising:
performing region of interest projection on the identified user physical information, wherein the identified user physical information corresponds to imaging data, received in form of an image or a video, of the user;
identifying region of interest based on the region of interest projection;
determining demographic information of the user based on the identified region of interest; and
identifying the plurality of protocol parameters for the medical examination based on the retrieved medical information and the determined demographic information.

3. The method of claim 1, further comprising:
performing region of interest projection on the identified user physical information, wherein the identified user physical information corresponds to imaging data, received in a form of an image or a video, of the user;
extracting the at least one of gesture, injury, and motion information from the identified user physical information based on the region of interest projection; and
selecting the one or more medical examination protocols based on the extracted at least one of gesture, injury, and motion information.

4. The method of claim 1, wherein the processing further comprises:
extracting, from the received input data, one or more protocol parameters and features corresponding to the one or more protocol parameters;
identifying the at least one protocol parameter among the plurality of protocol parameters based on the extracted features; and
controlling the change in the at least one protocol parameter with respect to the extracted one or more protocol parameters, based on at least one of the priority level and suggested change falling within the pre-defined range of the at least one protocol parameter.

5. The method of claim 4, wherein the processing further comprises:
when the priority level is indicated high, changing the at least one protocol parameter with respect to the extracted one or more protocol parameters whether the suggested change falls within the pre-defined range or outside the pre-defined range; and
when the priority level is indicated low, controlling the change in the at least one protocol parameter with respect to the extracted one or more protocol parameters based on the medical information whether the suggested change falls within the pre-defined range or outside the pre-defined range.

6. An artificial intelligence (AI) based protocol selection system for optimizing a medical imaging examination of a user, comprising:
an imaging and sensing unit configured to identify user physical information, wherein the user physical information includes at least one of gesture, height, weight, injury, motion, and structure information of the user;
one or more processor operatively coupled to the imaging and sensing unit, the one or more processor is configured to:
retrieve medical information of the user, wherein the user medical information corresponds to at least one of a medical history and current medical assessment of the user;
select one or more medical examination protocols based on at least a part of the identified user physical information; and
identify a plurality of protocol parameters for the medical examination, based on the retrieved user medical information and the selected one or more medical examination protocols;
a receiving unit operatively coupled to the one or more processor and configured to receive input data tagged with priority level, wherein the received input data is related to a change in at least one protocol parameter among the plurality of protocol parameters,
wherein the one or more processor is further configured to process the received input to change the at least one protocol parameter based on at least one of the priority level and a pre-defined range of the at least one protocol parameter; and
a transmitting unit operatively coupled to the one or more processor and configured to provide the plurality of protocol parameters based on the processed input data to a medical imaging apparatus for performing the medical imaging examination of the user.

7. The system of claim 6, wherein the one or more processor is further configured to:
perform region of interest projection on the identified user physical information, wherein the identified user physical information corresponds to imaging data, received in form of an image or a video, of the user;
identify region of interest based on the region of interest projection;
determine demographic information of the user based on the identified region of interest; and
identify the plurality of protocol parameters for the medical examination based on the retrieved medical information and the determined demographic information.

8. The system of claim 6, wherein the one or more processor is further configured to:
perform region of interest projection on the identified user physical information, wherein the identified user physical information corresponds to imaging data, received in a form of an image or a video, of the user;
extract the at least one of gesture, injury, and motion information from the identified user physical information based on the region of interest projection; and
select the one or more medical examination protocols based on the extracted at least one of gesture, injury, and motion information.

9. The system of claim 6, wherein the one or more processor is further configured to:
extract, from the received input data, one or more protocol parameters and features corresponding to the extracted one or more protocol parameters;
identify the at least one protocol parameter among the plurality of protocol parameters based on the extracted features; and
control the change in the at least one protocol parameter with respect to the extracted one or more protocol parameters, based on at least one of the priority level and suggested change falling within the pre-defined range of the at least one protocol parameters.

10. The system of claim 9, wherein the one or more processor is further configured to:
when the priority level is indicated high, change the at least one parameter with respect to the extracted one or more protocol parameters whether the suggested change falls within the pre-defined range or outside the pre-defined range; and
when the priority level is indicated low, control the change in the at least one protocol parameter with respect to the extracted one or more protocol parameters based on the medical information whether the suggested change falls within the pre-defined range or outside the pre-defined range.
